SEQUENCE LISTING (33 sequences)

SEQ ID NO: 1 - protein, 15 aa, Artificial Sequence
  Synthetic polypeptide
RASKGVSTSGYSYLH

SEQ ID NO: 2 - protein, 7 aa, Artificial Sequence
  Synthetic polypeptide
LASYLES

SEQ ID NO: 3 - protein, 9 aa, Artificial Sequence
  Synthetic polypeptide
QHSRDLPLT

SEQ ID NO: 4 - protein, 111 aa, Artificial Sequence [sequence omitted]

SEQ ID NO: 5 - protein, 218 aa, Artificial Sequence
  Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  Synthetic polypeptide
NYYMY

SEQ ID NO: 7 - protein, 17 aa, Artificial Sequence
  Synthetic polypeptide
GINPSNGGTNFNEKFKN

SEQ ID NO: 8 - protein, 11 aa, Artificial Sequence
  Synthetic polypeptide
RDYRFDMGFDY

SEQ ID NO: 9 - protein, 120 aa, Artificial Sequence [sequence omitted]

SEQ ID NO: 10 - protein, 447 aa, Artificial Sequence
  Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence
  Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 440 aa, Artificial Sequence
  Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 17 aa, Artificial Sequence
  Synthetic polypeptide
KSSQSLLHTSTRKNYLA

SEQ ID NO: 14 - protein, 7 aa, Artificial Sequence
  Synthetic polypeptide
WASTRES

SEQ ID NO: 15 - protein, 8 aa, Artificial Sequence
  Synthetic polypeptide
KQSYDVVT

SEQ ID NO: 16 - protein, 112 aa, Artificial Sequence
  Synthetic polypeptide
DIVMSQSPSSLAVSAGEKVTMTCKSSQSLLHTSTRKNYLAWYQQKPGQSPKLLIYWASTRESGVPDRFTGSGSGTDFTLTISSVQAEDLAVYYCKQSYDVVTFGAGTKLELK

SEQ ID NO: 17 - protein, 5 aa, Artificial Sequence
  Synthetic polypeptide
SYWIH

SEQ ID NO: 18 - protein, 10 aa, Artificial Sequence
  Synthetic polypeptide
GYTFTSYWIH

SEQ ID NO: 19 - protein, 17 aa, Artificial Sequence
  Synthetic polypeptide
YINPSSGYHEYNQKFID

SEQ ID NO: 20 - protein, 13 aa, Artificial Sequence
  Synthetic polypeptide
SGWLIHGDYYFDF

SEQ ID NO: 21 - protein, 122 aa, Artificial Sequence
  Synthetic polypeptide
  MISC_FEATURE (1)..(1): wherein X = Q or pE (pyro-glutamate)
XVHLQQSGAELAKPGASVKMSCKASGYTFTSYWIHWIKQRPGQGLEWIGYINPSSGYHEYNQKFIDKATLTADRSSSTAYMHLTSLTSEDSAVYYCARSGWLIHGDYYFDFWGQGTTLTVSS

SEQ ID NO: 22 - protein, 218 aa, Artificial Sequence
  Synthetic polypeptide
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 446 aa, Artificial Sequence
  Synthetic polypeptide
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 111 aa, Artificial Sequence
  Synthetic polypeptide
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIK

SEQ ID NO: 25 - protein, 119 aa, Artificial Sequence
  Synthetic polypeptide
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSS

SEQ ID NO: 26 - protein, 15 aa, Artificial Sequence
  Synthetic polypeptide
KASQSLDYEGDSDMN

SEQ ID NO: 27 - protein, 7 aa, Artificial Sequence
  Synthetic polypeptide
GASNLES

SEQ ID NO: 28 - protein, 9 aa, Artificial Sequence
  Synthetic polypeptide
QQSTEDPRT

SEQ ID NO: 29 - protein, 5 aa, Artificial Sequence
  Synthetic polypeptide
DYNVD

SEQ ID NO: 30 - protein, 17 aa, Artificial Sequence
  Synthetic polypeptide
DINPNDGGTIYAQKFQE

SEQ ID NO: 31 - protein, 10 aa, Artificial Sequence
  Synthetic polypeptide
NYRWFGAMDH

SEQ ID NO: 32 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 33 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

(19) United States
(12) Patent Application Publication
(10) Pub. No.: US 2021/0317214 A1
(43) Pub. Date: Oct. 14, 2021

(54) COMBINATION THERAPY OF A PD-1 ANTAGONIST AND LAG3 ANTAGONIST FOR TREATING PATIENTS WITH NON-MICROSATELLITE INSTABILITY-HIGH OR PROFICIENT MISMATCH REPAIR COLORECTAL CANCER

(71) Applicant: Merck Sharp & Dohme Corp., Rahway, NJ (US)

(72) Inventors: Elliot K. Chartash, Basking Ridge, NJ (US); Terrill K. McClanahan, Sunnyvale, CA (US); Jane Anne Healy, Hoboken, NJ (US)

(73) Assignee: Merck Sharp & Dohme Corp., Rahway, NJ (US)

(21) Appl. No.: 17/274,531

(22) PCT Filed: Sep. 9, 2019
(86) PCT No.: PCT/US2019/050122

§ 371 Date: Mar. 9, 2021

Related U.S. Application Data
(60) Provisional application No. 62/730,772, filed on Sep. 13, 2018.
(60) Provisional application No. 62/755,844, filed on Nov. 5, 2018.

(51) Int. Cl. C07K 16/28; A61P 35/00

(52) U.S. Cl. CPC C07K 16/2818; C07K 16/2803; A61K 2039/507; C07K 2317/24; C07K 2317/21; A61P 35/00

(57) ABSTRACT

The present disclosure describes combination therapies comprising an antagonist of Programmed Death 1 receptor (PD-1) and a Lymphocyte-Activation Gene 3 (LAG3) antagonist, and the use of the combination therapies for the treatment of non-microsatellite instability-high (non-MSI-H) or proficient mismatch repair (pMMR) colorectal cancer.

### FIELD OF THE INVENTION

The present invention relates to combination therapies useful for the treatment of cancer. In particular, the invention relates to a combination therapy which comprises an antagonist of a Programmed Death 1 protein (PD-1) and an antagonist of Lymphocyte-Activation Gene 3 (LAG3).

### BACKGROUND OF THE INVENTION

PD-1 is recognized as an important molecule in immune regulation and the maintenance of peripheral tolerance. PD-1 is moderately expressed on naive T, B and NKT cells and up-regulated by T/B cell receptor signaling on lymphocytes, monocytes and myeloid cells (1).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (2-13). Similarly, PD-1 expression on tumor infiltrating lymphocytes was found to mark dysfunctional T cells in breast cancer and melanoma (14-15) and to correlate with poor prognosis in renal cancer (16). Thus, it has been proposed that PD-L1 expressing tumor cells interact with PD-1 expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 have been approved for treating cancer. Pembrolizumab is a potent humanized immunoglobulin G4 (IgG4) mAb with high specificity of binding to the programmed cell death 1 (PD 1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1. Keytruda® (pembrolizumab) is indicated for the treatment of patients across a number of indications.

Lymphocyte-Activation Gene 3 (LAG3) is an inhibitory immune modulatory receptor that regulates effector T cell homeostasis, proliferation, and activation, and has a role in the suppressor activity of regulatory T cells (Tregs). LAG3 is expressed on activated CD8+ and CD4+ T cells, Tregs and the Trl regulatory T-cell population, as well as on natural killer cells and a subset of tolerogenic plasmacytoid dendritic cells. Because of its proposed role on both effector T cells and Tregs, LAG3 is one of several immune checkpoint molecules where simultaneous blockade of both cell populations has the potential to enhance antitumor immunity.

LAG3 is structurally related to cluster of differentiation (CD) 4 and a member of the immunoglobulin (Ig) superfamily. Like CD4, its ligand is major histocompatibility complex (MHC) Class II molecules. Interaction with its ligand leads to dimerization and signal transduction resulting in altered T-cell activation. Following T-cell activation, LAG3 is transiently expressed on the cell surface. A large proportion of LAG3 molecules are found in intracellular stores and can be rapidly translocated to the cell membrane upon T-cell activation. LAG3 expression is regulated at the cell surface by extracellular cleavage to yield a soluble form of LAG3 (sLAG 3), which can be detected in serum. Expression of LAG3 is tightly regulated and represents a self-limiting mechanism to counter uncontrolled T-cell activity.

In the United States (US), CRC is the third most common diagnosed cancer and the third leading cause of cancer death in both men and women. The American Cancer Society estimated that 132,640 people will be diagnosed with CRC and 49,700 people will die from the disease in 2015. Despite recent advances, the intent of treatment for most of mCRC participants is palliative with few patients achieving long-term survival (5-year survival rate of 13.5%). Current standard of care (SOC) treatments for mCRC in the early-line setting include chemotherapy based on fluoropyrimidine, oxaliplatin, and irinotecan used in combination or sequentially, with option for monoclonal antibodies targeting vascular endothelial growth factor (VEGF) (e.g., bevacizumab, ziv-aflibercept) or its receptors (eg, ramucirumab), and in patients with Ras wild type tumors, monoclonal antibodies targeting the epidermal growth factor (EGF) receptor (e.g., cetuximab, panitumumab). However, treatment options for heavily pre-treated patients beyond the second-line setting are especially limited and associated toxicities can be severe.

Lynch syndrome is a genetic disorder defined by defective mismatch repair that increases susceptibility to various cancer types, including CRC. Diagnosis can be confirmed with one of two biologically distinct but diagnostically equivalent tests, a) IHC characterization of Mismatch Repair (MMR) protein expression and b) PCR of genetic microsatellite markers in tumor tissue. The results of MMR IHC and PCR-based MSI testing have been shown to be largely concordant (97.80% concordance, exact 95% CI: 96.27-98.82). Bartley et. al. Cancer Prev Res (Phila) 2012; 5:320-327. Anti-cancer activity in the colorectal cancer (CRC) population with anti-PD-1 therapies including pembrolizumab has been limited to cancers with the deficient Mismatch Repair (dMMR)/Microsatellite Instability High (MSI-H) phenotype, which represents a minority (˜5%) of the Stage IV metastatic colorectal cancer (mCRC) population. By contrast, anti-PD-1 therapy has demonstrated little to no benefit in mCRC tumors that are non-MSI-H or have proficient Mismatch Repair (pMMR). MSI-H colorectal tumors are found predominantly in the proximal colon, and are associated with a less aggressive clinical course than are stage-matched Microsatellite Instability Low (MSI-L) or Microsatellite Stable (MSS) tumors. Since approximately 95% of mCRC patients have tumors that are non-MSI-H or pMMR, there is a need to develop combination regimens that would provide durable clinical benefit. While high response rates are reported in previously untreated mCRC population with current standard chemotherapeutic therapies, durability of clinical benefit is limited. Furthermore, treatment options for heavily pre-treated patients beyond the second-line setting are limited, and associated toxicities can be severe. Regorafenib and TAS-102 are accepted third line standard of care (SOC) therapies for patients with mCRC that is non MSI-H/pMMR. These therapies are approved for mCRC patients who have been treated with fluoropyrimidine-, irinotecan-, oxaliplatin-containing chemotherapies, anti-VEGF or an anti-EGFR agent (if KRAS wild-type). Despite regulatory approval, regorafenib and TAS-102 offer minimal benefits as ORR is ≤2% for both agents. Minimal durability of clinical benefit is evidenced by a 6-month PFS rate of ˜15%. Clearly, there is a high unmet medical need in developing novel combination regimens to improve the clinical outcome for patients with non-MSI-H/pMMR CRC.

### SUMMARY OF THE INVENTION

In one embodiment, the invention provides a method for treating non-microsatellite instablility-high (non-MSI-H) or proficient mismatch repair (pMMR) colorectal cancer (CRC) in an individual comprising administering to the individual a combination therapy which comprises a PD-1 antagonist and a LAG3 antagonist. In one embodiment, the PD-1 antagonist and LAG3 antagonist are co-formulated. In another embodiment, the PD-1 antagonist and LAG3 antagonist are co-administered. In a further embodiment, the tumor cells of the individual is PD-L1 expression positive. In one embodiment, the PD-1 antagonist is an anti-PD-1 antibody that blocks the binding of PD-1 to PD-L1 and PD-L2. In another embodiment, the LAG3 antagonist is an anti-LAG3 antibody that blocks the binding of LAG3 to MHC Class II molecules.

### BRIEF DESCRIPTION OF THE DRAWINGS

FIG. 1 CT scan of patient with non-MSI-H colorectal cancer before (left) and after (right) treatment with 21 mg anti-LAG3 antibody Ab6 and pembrolizumab. The patient received 5 prior lines of chemotherapy, no prior anti-PD-1 or anti-PD-L1 therapy. The patient had a partial response with 45% reduction in tumor volume. There was also tumor volume reduction in lung lesions and lymph nodes, and stable presacral mass. The response is ongoing at 13.5 months.

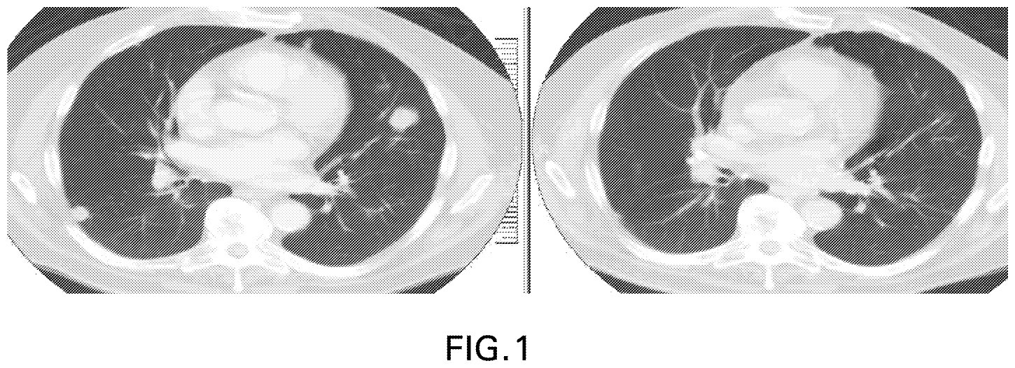

FIG. 2 Waterfall plot of subjects with best target lesion change from baseline based on investigator assessment per RECIST 1.1 FAS population in the colorectal cancer cohort (Part B) using the PD-L1 IHC Combined Positive (MIDS+TPS) (Positive: TPS>=1 OR MIDS>=2) score. Each bar represents an individual subject. Greater than a 30% decrease in tumor size from baseline (Y-axis) is considered a response; changes between a 30% decrease and a 20% increase is considered stable disease; changes greater than a 20% increase is considered progressive disease. PD-L1 positive or negative tumors are indicated. Tumor samples with less than 100 tumor cells cannot be interpreted. “Empty” indicate missing data.

FIG. 3 Waterfall plot of subjects with best target lesion change from baseline based on investigator assessment per RECIST 1.1 FAS population in the colorectal cancer cohort (Part B) using the PD-L1 IHC MIDS score. Each bar represents an individual subject. Greater than a 30% decrease in tumor size from baseline (Y-axis) is considered a response; changes between a 30% decrease and a 20% increase is considered stable disease; changes greater than a 20% increase is considered progressive disease. PD-L1 positive or negative tumors are indicated. Tumor samples with less than 100 tumor cells cannot be interpreted. “Empty” indicate missing data.

FIG. 4 Waterfall plot of subjects with best target lesion change from baseline based on investigator assessment per RECIST 1.1 FAS population in the colorectal cancer expansion cohort (Part B) using the PD-L1 IHC Combined Positive (MIDS+TPS) score (CPS). Each bar represents an individual subject. Greater than a 30% decrease in tumor size from baseline (Y-axis) is considered a response; changes between a 30% decrease and a 20% increase is considered stable disease; changes greater than a 20% increase is considered progressive disease. Tumor samples with CPS>=1 or <1 are indicated. Tumor samples with less than 100 tumor cells cannot be interpreted.

### DETAILED DESCRIPTION

Abbreviations. Throughout the detailed description and examples of the invention the following abbreviations will be used:
BOR Best overall response
BID One dose twice daily
CBR Clinical Benefit Rate
CDR Complementarity determining region
CHO Chinese hamster ovary
CR Complete Response
DCR Disease Control Rate
DFS Disease free survival
DLT Dose limiting toxicity
DOR Duration of Response
DSDR Durable Stable Disease Rate
FFPE Formalin-fixed, paraffin-embedded
FR Framework region
IgG Immunoglobulin G
IHC Immunohistochemistry or immunohistochemical
irRC Immune related response criteria
IV Intravenous
MTD Maximum tolerated dose
NCBI National Center for Biotechnology Information
NCI National Cancer Institute
ORR Objective response rate
OS Overall survival
PD Progressive disease
PD-1 Programmed Death 1
PD-L1 Programmed Cell Death 1 Ligand 1
PD-L2 Programmed Cell Death 1 Ligand 2
PFS Progression free survival
PR Partial response
Q2W One dose every two weeks
Q3W One dose every three weeks
QD One dose per day
RECIST Response Evaluation Criteria in Solid Tumors
SD Stable disease
VH Immunoglobulin heavy chain variable region
VK Immunoglobulin kappa light chain variable region

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

As used herein, an “Ab6 variant” means a monoclonal antibody which comprises heavy chain and light chain sequences that are substantially identical to those in Ab6 (as described below and in WO2016028672, incorporated by reference in its entirety), except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, Ab6 and a Ab6 variant comprise identical CDR sequences, but differ from each other due to having a conservative amino acid substitution at no more than three or six other positions in their full length light and heavy chain sequences, respectively. An Ab6 variant is substantially the same as Ab6 with respect to the following properties: binding affinity to human LAG3 and ability to block the binding of human LAG3 to human MHC Class II.

“Administration” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “subject” includes any organism, preferably an animal, more preferably a mammal (e.g., rat, mouse, dog, cat, rabbit) and most preferably a human.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Co-administration” as used herein for agents such as the PD-1 antagonist or LAG3 antagonist means that the agents are administered so as to have overlapping therapeutic activities, and not necessarily that the agents are administered simultaneously to the subject. The agents may or may not be in physical combination prior to administration. In an embodiment, the agents are administered to a subject simultaneously or at about the same time. For example, the anti-PD-1 antibody and anti-LAG3 drug products contained in separate vials, when in liquid solution, may be mixed into the same intravenous infusion bag or injection device, and administered simultaneously to the patient.

“Co-formulated” or “co-formulation” or “coformulation” or “coformulated” as used herein refers to at least two different antibodies or antigen binding fragments thereof which are formulated together and stored as a combined product in a single vial or vessel (for example an injection device) rather than being formulated and stored individually and then mixed before administration or separately administered. In one embodiment, the co-formulation contains two different antibodies or antigen binding fragments thereof.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Anti-tumor response” when referring to a cancer patient treated with a therapeutic regimen, such as a combination therapy described herein, means at least one positive therapeutic effect, such as for example, reduced number of cancer cells, reduced tumor size, reduced rate of cancer cell infiltration into peripheral organs, reduced rate of tumor metastasis or tumor growth, or progression free survival. Positive therapeutic effects in cancer can be measured in a number of ways (See, W. A. Weber, J. Null. Med. 50:1S-10S (2009); Eisenhauer et al., supra). In some embodiments, an anti-tumor response to a combination therapy described herein is assessed using RECIST 1.1 criteria, bidimentional irRC or unidimensional irRC. In some embodiments, an anti-tumor response is any of SD, PR, CR, PFS, or DFS.

“Bidimensional irRC” refers to the set of criteria described in Wolchok J D, et al. Guidelines for the evaluation of immune therapy activity in solid tumors: immune-related response criteria. Clin Cancer Res. 2009; 15(23):7412-7420. These criteria utilize bidimensional tumor measurements of target lesions, which are obtained by multiplying the longest diameter and the longest perpendicular diameter (cm²) of each lesion.

“Biotherapeutic agent” means a biological molecule, such as an antibody or fusion protein, that blocks ligand/receptor signaling in any biological pathway that supports tumor maintenance and/or growth or suppresses the anti-tumor immune response. Classes of biotherapeutic agents include, but are not limited to, antibodies to VEGF, EGFR, Her2/neu, other growth factor receptors, CD20, CD40, CD-40L, CTLA-4, OX-40, 4-1BB, and ICOS.

“CBR” or “Clinical Benefit Rate” means CR+PR+durable SD

“CDR” or “CDRs” as used herein means complementarity determining region(s) in a immunoglobulin variable region, defined using the Kabat numbering system, unless otherwise indicated.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods of the present invention include cytostatic and/or cytotoxic agents.

“Chothia” as used herein means an antibody numbering system described in Al-Lazikani et al., JMB 273:927-948 (1997).

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a PD-1 antagonist that consists essentially of a recited amino acid sequence may also include one or more amino acids, including substitutions of one or more amino acid residues, which do not materially affect the properties of the binding compound.

“DCR” or “Disease Control Rate” means CR+PR+SD.

“Diagnostic anti-PD-L monoclonal antibody” means a mAb which specifically binds to the mature form of the designated PD-L (PD-L1 or PDL2) that is expressed on the surface of certain mammalian cells. A mature PD-L lacks the presecretory leader sequence, also referred to as leader peptide The terms “PD-L” and “mature PD-L” are used interchangeably herein, and shall be understood to mean the same molecule unless otherwise indicated or readily apparent from the context.

As used herein, a diagnostic anti-human PD-L1 mAb or an anti-hPD-L1 mAb refers to a monoclonal antibody that specifically binds to mature human PD-L1. A mature human PD-L1 molecule consists of amino acids 19-290 of the following sequence:

(SEQ ID NO: 32)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKITTINSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET.

Specific examples of diagnostic anti-human PD-L1 mAbs useful as diagnostic mAbs for immunohistochemistry (IHC) detection of PD-L1 expression in formalin-fixed, paraffin-embedded (FFPE) tumor tissue sections are antibody 20C3 and antibody 22C3, which are described in WO2014/100079. Another anti-human PD-L1 mAb that has been reported to be useful for IHC detection of PD-L1 expression in FFPE tissue sections (Chen, B. J. et al., Clin Cancer Res 19: 3462-3473 (2013)) is a rabbit anti-human PD-L1 mAb publicly available from Sino Biological, Inc. (Beijing, P.R. China; Catalog number 10084-R015).

TABLE 2
Characteristics of Monoclonal Antibody MEB037.22C3 (22C3)

| | | |
|---|---|---|
| | | SEQ ID |
| Antibody Feature | Amino Acid Sequence | NO |

Light Chain

| | | |
|---|---|---|
| CDRL1 | KSSQSLLHTSTRKNYLA | 13 |
| CDRL2 | WASTRES | 14 |
| CDRL3 | KQSYDVVT | 15 |
| Mature Variable | DIVMSQSPSSLAVSAGEKVTMTCKSSQSLLHTSTRKNYLAWYQ | 16 |
| Region | QKPGQSPKLLIYWASTRESGVPDRFTGSGSGTDFTLTISSVQAE | |
| | DLAVYYCKQSYDVVTFGAGTKLELK | |

Heavy Chain

| | | |
|---|---|---|
| CDRH1 Kabat Def'n | SYWIH | 17 |
| CDRH1 Chothia Def'n | GYTFTSYWIH | 18 |
| CDRH2 | YINPSSGYHEYNQKFID | 19 |
| CDRH3 | SGWLIHGDYYFDF | 20 |
| Mature Variable | XVHLQQSGAELAKPGASVKMSCKASGYTFTSYWIHWIKQRPG | 21 |
| Region | QGLEWIGYINPSSGYHEYNQKFIDKATLTADRSSSTAYMHLTSL | |
| | TSEDSAVYYCARSGWLIHGDYYFDFWGQGTTLTVSS, | |
| | wherein X = Q or pE (pyro-glutamate) | |

“PD-L1” or “PD-L2” expression as used herein means any detectable level of expression of the designated PD-L protein on the cell surface or of the designated PD-L mRNA within a cell or tissue. PD-L protein expression may be detected with a diagnostic PD-L antibody in an IHC assay of a tumor tissue section or by flow cytometry. Alternatively, PD-L protein expression by tumor cells may be detected by PET imaging, using a binding agent (e.g., antibody fragment, affibody and the like) that specifically binds to the desired PD-L target, e.g., PD-L1 or PD-L2. Techniques for detecting and measuring PD-L mRNA expression include RT-PCR, realtime quantitative RT-PCR, RNAseq, and the Nanostring platform (J. Clin. Invest. 2017; 127(8):2930-2940).

Several approaches have been described for quantifying PD-L1 protein expression in IHC assays of tumor tissue sections. See, e.g., Thompson, R. H., et al., PNAS 101 (49); 17174-17179 (2004); Thompson, R. H. et al., Cancer Res. 66:3381-3385 (2006); Gadiot, J., et al., Cancer 117:2192-2201 (2011); Taube, J. M. et al., Sci Transl Med 4, 127ra37 (2012); and Toplian, S. L. et al., New Eng. J Med. 366 (26): 2443-2454 (2012). See US 20170285037 which describes Hematoxylin and Eosin staining used by the pathologist.

One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression if it is at least 1% of total tumor cells.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the AIS is ≥5.

The level of PD-L mRNA expression may be compared to the mRNA expression levels of one or more reference genes that are frequently used in quantitative RT-PCR.

In some embodiments, a level of PD-L1 expression (protein and/or mRNA) by malignant cells and/or by infiltrating immune cells within a tumor is determined to be “overexpressed” or “elevated” based on comparison with the level of PD-L1 expression (protein and/or mRNA) by an appropriate control. For example, a control PD-L1 protein or mRNA expression level may be the level quantified in nonmalignant cells of the same type or in a section from a matched normal tissue. In some preferred embodiments, PD-L1 expression in a tumor sample is determined to be elevated if PD-L1 protein (and/or PD-L1 mRNA) in the sample is at least 10%, 20%, or 30% greater than in the control.

“Tumor proportion score (TPS)” refers to the percentage of tumor cells expressing PD-L1 on the cell membrane at any intensity (weak, moderate or strong). Linear partial or complete cell membrane staining is interpreted as positive for PD-L1.

“Mononuclear inflammatory density score (MIDS)” refers to the ratio of the number of PD-L1 expressing mononuclear inflammatory cells (MIC) infiltrating or adjacent to the tumor (small and large lymphocytes, monocytes, and macrophages within the tumor nests and the adjacent supporting stroma) compared to the total number of tumor cells. The MIDS is recorded at a scale from 0 to 4 with 0=none; 1=present, but less than one MIC for every 100 tumor cells (<1%); 2=at least one MIC for every 100 tumor cells, but less than one MIC per 10 tumor cells (1-9%), 3=at least one MIC for every 10 tumor cells, but fewer MIC's than tumor cells (10-99%), 4=at least as many MIC's as tumor cells (≥100%).

“Combined positive score (CPS)” refers to the ratio of the number of PD-L1 positive tumor cells and PD-L1 positive mononuclear inflammatory cells (MIC) within the tumor nests and the adjacent supporting stroma (numerator) compared to the total number of tumor cells (denominator; i.e., the number of PD-L1 positive and PD-L1 negative tumor cells). PD-L1 expression at any intensity is considered positive, i.e., weak (1+), moderate (2+), or strong (3+).

“PD-L1 expression positive” refers to a Tumor Proportion Score, Mononuclear Inflammatory Density Score or Combined Positive Score of at least 1%; AIS is ≥5; or elevated level of PD-L1 expression (protein and/or mRNA) by malignant cells and/or by infiltrating immune cells within a tumor compared to an appropriate control.

“DSDR” or “Durable Stable Disease Rate” means SD for ≥23 weeks.

“Framework region” or “FR” as used herein means the immunoglobulin variable regions excluding the CDR regions.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“LAG3 antagonist” means any chemical compound or biological molecule that blocks binding of LAG3 expressed on an immune cell (T cell, Tregs, or NK cell etc.) to MHC Class II molecules. Human LAG3 comprises the amino acid sequence:

(SEQ ID NO: 33)
MWEAQFLGLL FLQPLWVAPV KPLQPGAEVP VVWAQEGAPA
QLPCSPTIPL QDLSLLRRAG VTWQHQPDSG PPAAAPGHPL
APGPHPAAPS SWGPRPRRYT VLSVGPGGLR SGRLPLQPRV
QLDERGRQRG DFSLWLRPAR RADAGEYRAA VHLRDRALSC
RLRLRLGQAS MTASPPGSLR ASDWVILNCS FSRPDRPASV
HWFRNRGQGR VPVRESPHHH LAESFLFLPQ VSPMDSGPWG
CILTYRDGFN VSIMYNLTVL GLEPPTPLTV YAGAGSRVGL
PCRLPAGVGT RSFLTAKWTP PGGGPDLLVT GDNGDFTLRL
EDVSQAQAGT YTCHIHLQEQ QLNATVTLAI ITVTPKSFGS
PGSLGKLLCE VTPVSGQERF VWSSLDTPSQ RSFSGPWLEA
QEAQLLSQPW QCQLYQGERL LGAAVYFTEL SSPGAQRSGR
APGALPAGHL LLFLILGVLS LLLLVTGAFG FHLWRRQWRP
RRFSALEQGI HPPQAQSKIE ELEQEPEPEP EPEPEPEPEP EPEQL;
see also Uniprot accession no. P18627.

“Microsatellite instability (MSI)” refers to the form of genomic instability associated with defective DNA mismatch repair in tumors. See Boland et al., Cancer Research 58, 5258-5257, 1998. In one embodiment, MSI analysis can be carried out using the five National Cancer Institute (NCI) recommended microsatellite markers BAT25 (GenBank accession no. 9834508), BAT26 (GenBank accession no. 9834505), D5S346 (GenBank accession no. 181171), D2S123 (GenBank accession no. 187953), D175250 (GenBank accession no. 177030). Additional markers for example, BAT40, BAT34C4, TGF-β-RII and ACTC can be used. Commercially available kits for MSI analysis include, for example, the Promega MSI multiplex PCR assay.

“High frequency microsatellite instability” or “microsatellite instability-high (MSI-H)” refers to if two or more of the five NCI markers show instability or ≥30-40% of the total markers demonstrate instability (i.e. have insertion/deletion mutations).

“Low frequency microsatellite instability” or “microsatellite instability-low (MSI-L)” refers to if one of the five NCI markers show instability or <30-40% of the total markers exhibit instability (i.e. have insertion/deletion mutations).

“Non-MSI-H colorectal cancer” as used herein refers to microsatellite stable (MSS) and low frequency MSI (MSI-L) colorectal cancer.

“Microsatellite Stable (MSS)” refers to if none of the five NCI markers show instability (i.e. have insertion/deletion mutations)

“Proficient mismatch repair (pMMR) colorectal cancer” refers to normal expression of MMR proteins (MLH1, PMS2, MSH2, and MSH6) in a CRC tumor specimen by IHC. Commercially available kits for MMR analysis include theVentana MMR IHC assay.

“Mismatch repair deficient (dMMR) colorectal cancer” refers to low expression of one or more MMR protein(s) (MLH1, PMS2, MSH2, and MSH6) in a CRC tumor specimen by IHC.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Non-responder patient”, when referring to a specific anti-tumor response to treatment with a combination therapy described herein, means the patient did not exhibit the anti-tumor response.

“ORR” or “objective response rate” refers in some embodiments to CR+PR, and ORR_{(week 24)} refers to CR and PR measured using irRECIST in each patient in a cohort after 24 weeks of anti-cancer treatment.

“Patient” or “subject” refers to any single subject for which therapy is desired or that is participating in a clinical trial, epidemiological study or used as a control, including humans and mammalian veterinary patients such as cattle, horses, dogs, and cats.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

As used herein, a “pembrolizumab variant” means a monoclonal antibody which comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g, the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“RECIST 1.1 Response Criteria” as used herein means the definitions set forth in Eisenhauer et al., E. A. et al., Eur. J Cancer 45:228-247 (2009) for target lesions or nontarget lesions, as appropriate based on the context in which response is being measured.

“Responder patient” when referring to a specific anti-tumor response to treatment with a combination therapy described herein, means the patient exhibited the anti-tumor response.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Tissue Section” refers to a single part or piece of a tissue sample, e.g., a thin slice of tissue cut from a sample of a normal tissue or of a tumor.

“Treat” or “treating” cancer as used herein means to administer a combination therapy of a PD-1 antagonist and LAG3 antagonist to a subject having cancer, or diagnosed with cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, response to a combination therapy described herein is assessed using RECIST 1.1 criteria or irRC (bidimensional or unidimensional) and the treatment achieved by a combination of the invention is any of PR, CR, OR, PFS, DFS and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination of the invention is any of PR, CR, PFS, DFS, OR and OS that is assessed using RECIST 1.1 response criteria. The treatment regimen for a combination of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as the Student's t-test, the chit-test, the U-test according to Mann and Whitney, the Kruskal-Wallis test (H-test), Jonckheere-Terpstra-test and the Wilcoxon-test.

The terms “treatment regimen”, “dosing protocol” and “dosing regimen” are used interchangeably to refer to the dose and timing of administration of each therapeutic agent in a combination of the invention.

“Tumor” as it applies to a subject diagnosed with, or suspected of having, cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone marrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Unidimensional irRC refers to the set of criteria described in Nishino M, Giobbie-Hurder A, Gargano M, Suda M, Ramaiya N H, Hodi F S. Developing a Common Language for Tumor Response to Immunotherapy: Immune-related Response Criteria using Unidimensional measurements. Clin Cancer Res. 2013; 19(14):3936-3943). These criteria utilize the longest diameter (cm) of each lesion.

“Variable regions” or “V region” as used herein means the segment of IgG chains which is variable in sequence between different antibodies. Typically, it extends to Kabat residue 109 in the light chain and 113 in the heavy chain.

### PD-1 Antagonists and LAG3 Antagonists

PD-1 antagonists useful in the treatment method, medicaments and uses of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

Examples of mAbs that bind to human PD-1, and useful in the treatment method, medicaments and uses of the present invention, are described in U.S. Pat. Nos. 7,488,802, 7,521,051, 8,008,449, 8,354,509, 8,168,757, WO2004/004771, WO2004/072286, WO2004/056875, and US2011/0271358. Specific anti-human PD-1 mAbs useful as the PD-1 antagonist in the treatment method, medicaments and uses of the present invention include: pembrolizumab (also known as MK-3475), a humanized IgG4 mAb with the structure described in WHO Drug Information, Vol. 27, No. 2, pages 161-162 (2013) and which comprises the heavy and light chain amino acid sequences shown in Table 3; nivolumab (BMS-936558), a human IgG4 mAb with the structure described in WHO Drug Information, Vol. 27, No. 1, pages 68-69 (2013) and which comprises the heavy and light chain amino acid sequences shown in Table 3; the humanized antibodies h409A11, h409A16 and h409A17, which are described in WO2008/156712, and AMP-514, which is being developed by MedImmune.

Examples of mAbs that bind to human PD-L1, and useful in the treatment method, medicaments and uses of the present invention, are described in WO2013/019906, WO2010/077634 A1 and U.S. Pat. No. 8383796. Specific anti-human PD-L1 mAbs useful as the PD-1 antagonist in the treatment method, medicaments and uses of the present invention include MPDL3280A, BMS-936559, MEDI4736, MSB0010718C and an antibody which comprises the heavy chain and light chain variable regions of SEQ ID NO:24 and SEQ ID NO:21, respectively, of WO2013/019906.

Other PD-1 antagonists useful in the treatment method, medicaments and uses of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

In some preferred embodiments of the treatment method, medicaments and uses of the present invention, the PD-1 antagonist is a monoclonal antibody, or antigen binding fragment thereof, which comprises: (a) light chain CDRs SEQ ID NOs: 1, 2 and 3 and (b) heavy chain CDRs SEQ ID NOs: 6, 7 and 8.

In other preferred embodiments of the treatment method, medicaments and uses of the present invention, the PD-1 antagonist is a monoclonal antibody, or antigen binding fragment thereof, which specifically binds to human PD-1 and comprises (a) a heavy chain variable region comprising SEQ ID NO:9 or a variant thereof, and (b) a light chain variable region comprising SEQ ID NO:4 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another preferred embodiment of the treatment method, medicaments and uses of the present invention, the PD-1 antagonist is a monoclonal antibody which specifically binds to human PD-1 and comprises (a) a heavy chain comprising SEQ ID NO: 10 and (b) a light chain comprising SEQ ID NO:5.

In yet another preferred embodiment of the treatment method, medicaments and uses of the present invention, the PD-1 antagonist is a monoclonal antibody which specifically binds to human PD-1 and comprises (a) a heavy chain comprising SEQ ID NO: 12 and (b) a light chain comprising SEQ ID NO:11.

In all of the above treatment method, medicaments and uses, the PD-1 antagonist inhibits the binding of PD-L1 to PD-1, and preferably also inhibits the binding of PD-L2 to PD-1. In some embodiments of the above treatment method, medicaments and uses, the PD-1 antagonist is a monoclonal antibody, or an antigen binding fragment thereof, which specifically binds to PD-1 or to PD-L1 and blocks the binding of PD-L1 to PD-1. In one embodiment, the PD-1 antagonist is an anti-PD-1 antibody which comprises a heavy chain and a light chain, and wherein the heavy and light chains comprise the amino acid sequences in SEQ ID NO:10 and SEQ ID NO:5, respectively.

Table 3 below provides a list of the amino acid sequences of exemplary anti-PD-1 mAbs for use in the treatment method, medicaments and uses of the present invention.

TABLE 3
Exemplary PD-1 Antibody Sequences

| | | |
|---|---|---|
| Antibody | | SEQ ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSYLHWY | 4 |
| Region | QQKPGQAPRLLIYLASYLESGVPARFSGSGSGTDFTLTISS | |
| | LEPEDFAVYYCQHSRDLPLTFGGGTKVEIK | |
| Light | EIVLTQSPATLSLSPGERATLSCRASKGVSTSGYSYLHWY | 5 |
| Chain | QQKPGQAPRLLIYLASYLESGVPARFSGSGSGTDFTLTISS | |
| | LEPEDFAVYYCQHSRDLPLTFGGGTKVEIKRTVAAPSVFI | |
| | FPPSDEQLKSGTASVVCLLNNFYPREAKVQWKVDNALQS | |
| | GNSQESVTEQDSKDSTYSLSSTLTLSKADYEKHKVYACE | |
| | VTHQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNYYMYWV | 9 |
| Region | RQAPGQGLEWMGGINPSNGGTNFNEKFKNRVTLTTDSST | |
| | TTAYMELKSLQFDDTAVYYCARRDYRFDMGFDYWGQG | |
| | TTVTVSS | |
| Heavy | QVQLVQSGVEVKKPGASVKVSCKASGYTFTNYYMYWV | 10 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYFP | |
| | EPVTVSWNSGALTSGVHTFPAVLQSSGLYSLSSVVTVPSS | |
| | SLGTKTYTCNVDHKPSNTKVDKRVESKYGPPCPPCPAPE | |
| | FLGGPSVFLFPPKPKDTLMISRTPEVTCVVVDVSQEDPEV | |
| | QFNWYVDGVEVHNAKTKPREEQFNSTYRVVSVLTVLHQ | |
| | DWLNGKEYKCKVSNKGLPSSIEKTISKAKGQPREPQVYT | |
| | LPPSQEEMTKNQVSLTCLVKGFYPSDIAVEWESNGQPEN | |
| | NYKTTPPVLDSDGSFFLYSRLTVDKSRWQEGNVFSCSVM | |
| | HEALHNHYTQKSLSLSLGK | |

Nivolumab Light Chain

| | | |
|---|---|---|
| Light | EIVLTQSPATLSLSPGERATLSCRASQSVSSYLAWYQQKP | 11 |
| Chain | GQAPRLLIYDASNRATGIPARFSGSGSGTDFTLTISSLEPE | |
| | DFAVYYCQQSSNWPRTFGQGTKVEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |

Nivolumab Heavy Chain

| | | |
|---|---|---|
| Heavy | QVQLVESGGGVVQPGRSLRLDCKASGITFSNSGMHWVR | 12 |
| Chain | QAPGKGLEWVAVIWYDGSKRYYADSVKGRFTISRDNSK | |
| | NTLFLQMNSLRAEDTAVYYCATNDDYWGQGTLVTVSSA | |
| | STKGPSVFPLAPCSRSTSESTAALGCLVKDYFPEPVTVSW | |
| | NSGALTSGVHTFPAVLQSSGLYSLSSVVTVPSSSLGTKTY | |
| | TCNVDHKPSNTKVDKRVESKYGPPCPPCPAPEFLGGPSVF | |
| | LFPPKPKDTLMISRTPEVTCVVVDVSQEDPEVQFNWYVD | |
| | GVEVHNAKTKPREEQFNSTYRVVSVLTVLHQDWLNGKE | |
| | YKCKVSNKGLPSSIEKTISKAKGQPREPQVYTLPPSQEEM | |
| | TKNQVSLTCLVKGFYPSDIAVEWESNGQPENNYKTTPPV | |
| | LDSDGSFFLYSRLTVDKSRWQEGNVFSCSVMHEALHNH | |
| | YTQKSLSLSLGK | |

LAG3 antagonists useful in the treatment method, medicaments and uses of the present invention include a monoclonal antibody (mAb), or antigen binding fragment thereof, which specifically binds to LAG3. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

In one embodiment, the anti-LAG3 antibody is Ab6.

Ab6: a light chain immunoglobulin comprising the
amino acid sequence:
(SEQ ID NO: 22)
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC;
and
a heavy chain immunoglobulin comprising the amino
acid sequence:
(SEQ ID NO: 23)
QMQLVQSGPEVKKPGTSVKVSCKASGYTFTDYNVDWVRQARGQRLEWIGD
INPNDGGTIYAQKFQERVTITVDKSTSTAYMELSSLRSEDTAVYYCARNY
RWFGAMDHWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK;
or
a light chain immunoglobulin variable domain
comprising the amino acid sequence:
(SEQ ID NO: 24 (CDRs underscored))
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIK;
and
a heavy chain immunoglobulin variable domain
comprising the amino acid sequence:
(SEQ ID NO: 25 (CDRs underscored))
QMQLVQSGPEVKKPGTSVKVSCKASGYTFTDYNVDWVRQARGQRLEWIGD
INPNDGGTIYAQKFQERVTITVDKSTSTAYMELSSLRSEDTAVYYCARNY
RWFGAMDHWGQGTTVTVSS;
or comprising the CDRs:
CDR-L1:
(SEQ ID NO: 26)
KASQSLDYEGDSDMN;
CDR-L2:
(SEQ ID NO: 27)
GASNLES;
CDR-L3:
(SEQ ID NO: 28)
QQSTEDPRT;
CDR-H1:
(SEQ ID NO: 29)
DYNVD;
CDR-H2:
(SEQ ID NO: 30)
DINPNDGGTIYAQKFQE;
and
CDR-H3:
(SEQ ID NO: 31)
NYRWFGAMDH

In some preferred embodiments of the treatment method, medicaments and uses of the present invention, the LAG3 antagonist is a monoclonal antibody, or antigen binding fragment thereof, which comprises: (a) light chain CDRs SEQ ID NOs: 26, 27 and 28 and (b) heavy chain CDRs SEQ ID NOs: 29, 30 and 31.

In other preferred embodiments of the treatment method, medicaments and uses of the present invention, the LAG3 antagonist is a monoclonal antibody, or antigen binding fragment thereof, which specifically binds to human LAG3 and comprises (a) a heavy chain variable region comprising SEQ ID NO:25 or a variant thereof, and (b) a light chain variable region comprising SEQ ID NO:24 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another preferred embodiment of the treatment method, medicaments and uses of the present invention, the LAG3 antagonist is a monoclonal antibody which specifically binds to human LAG3 and comprises (a) a heavy chain comprising SEQ ID NO: 23 and (b) a light chain comprising SEQ ID NO:22. In another preferred embodiment of the treatment method, medicaments and uses of the present invention, the LAG3 antagonist is a monoclonal antibody which specifically binds to human LAG3 and comprises (a) a heavy chain variable region comprising SEQ ID NO: 25 and (b) a light chain variable region comprising SEQ ID NO:24.

Other Examples of mAbs that bind to human LAG3, and useful in the treatment method, medicaments and uses of the present invention, are relatlimab, IMP731, IMP701, anti-LAG3 antibodies disclosed in US2017101472. Other LAG3 antagonists useful in the treatment method, medicaments and uses of the present invention include an immunoadhesin that specifically binds to human LAG3, e.g., a fusion protein containing the extracellular LAG3 fused to a constant region such as an Fc region of an immunoglobulin molecule.

In one embodiment, the anti-PD-1 or anti-LAG3 antibody or antigen-binding fragment comprises a heavy chain constant region, e.g. a human constant region, such as γ1, γ2, γ3, or γ4 human heavy chain constant region or a variant thereof. In another embodiment, the anti-LAG3 antibody or antigen-binding fragment comprises a light chain constant region, e.g. a human light chain constant region, such as lambda or kappa human light chain region or variant thereof. By way of example, and not limitation, the human heavy chain constant region can be γ4 and the human light chain constant region can be kappa. In an alternative embodiment, the Fc region of the antibody is γ4 with a Ser228Pro mutation (Schuurman, J et. al., Mol. Immunol. 38: 1-8, 2001).

In some embodiments, different constant domains may be appended to humanized V_{L} and V_{H} regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized.

Although human IgG1 antibodies provide for long half-life and for effector functions, such as complement activation and antibody-dependent cellular cytotoxicity, such activities may not be desirable for all uses of the antibody. In such instances a human IgG4 constant domain, for example, may be used. The present invention includes the use of anti-PD-1 antibodies or anti-LAG3 antibodies and antigen-binding fragments thereof which comprise an IgG4 constant domain. In one embodiment, the IgG4 constant domain can differ from the native human IgG4 constant domain (Swiss-Prot Accession No. P01861.1) at a position corresponding to position 228 in the EU system and position 241 in the KABAT system, where the native Ser108 is replaced with Pro, in order to prevent a potential inter-chain disulfide bond between Cys106 and Cys109 (corresponding to positions Cys 226 and Cys 229 in the EU system and positions Cys 239 and Cys 242 in the KABAT system) that could interfere with proper intra-chain disulfide bond formation. See Angal et al. (1993) Mol. Imunol. 30:105. In other instances, a modified IgG1 constant domain which has been modified to increase half-life or reduce effector function can be used.

### Methods, Uses and Medicaments

In one aspect of the invention, the invention provides a method for treating non-MSI-H or pMMR colorectal cancer in an individual comprising co-administering to the individual a PD-1 antagonist and LAG3 antagonist. In another aspect of the invention, the invention provides a method for treating non-MSI-H or pMMR colorectal cancer in an individual comprising administering to the individual a composition comprising a PD-1 antagonist and a LAG3 antagonist.

In another embodiment, the invention provides a medicament comprising a PD-1 antagonist for use in combination with a LAG3 antagonist for treating non-MSI-H or pMMR colorectal cancer. In yet another embodiment, the invention provides a medicament comprising a LAG3 antagonist for use in combination with a PD-1 antagonist for treating non-MSI-H or pMMR colorectal cancer.

Other embodiments provide use of a PD-1 antagonist in the manufacture of a medicament for treating non-MSI-H or pMMR colorectal cancer in an individual when administered in combination with a LAG3 antagonist and use of a LAG3 antagonist in the manufacture of a medicament for treating non-MSI-H or pMMR colorectal cancer in an individual when administered in combination with a PD-1 antagonist.

In another embodiment, the invention provides a LAG3 antagonist for use in the treatment of non-MSI-H or pMMR colorectal cancer in an individual, wherein said use is in combination with a PD-1 antagonist. In a further embodiment, the invention provides a combination of a PD-1 antagonist and a LAG3 antagonist for use in treatment of a subject with non-MSI-H or pMMR colorectal cancer.

In a still further embodiment, the invention provides use of a PD-1 antagonist and a LAG3 antagonist in the manufacture of a medicament for treating non-MSI-H or pMMR colorectal cancer in an individual. In some embodiments, the medicaments comprise a kit, and the kit also comprises a package insert comprising instructions for using the PD-1 antagonist in combination with the LAG3 antagonist to treat non-MSI-H or pMMR colorectal cancer in an individual.

In the foregoing methods, medicaments and uses, in one embodiment, the PD-1 antagonist and LAG3 antagonist are co-formulated. In another embodiment, the PD-1 antagonist and LAG3 antagonist are co-administered. The treatment may further comprise administration of mFOLFOX7 (Leucovorin (Calcium Folinate), Fluorouracil (5-FU), Oxaliplatin) or FOLFIRI (Leucovorin (Calcium Folinate), Fluorouracil (5-FU), Irinotecan Hydrochloride). In one embodiment, the PD-1 antagonist is an anti-PD-1 antibody that blocks the binding of PD-1 to PD-L1 and PD-L2. In one embodiment, the LAG3 antagonist is an anti-LAG3 antibody that blocks the binding of LAG3 to MHC Class II.

The combination therapy may also comprise one or more additional therapeutic agents. The additional therapeutic agent may be, e.g., a chemotherapeutic, a biotherapeutic agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF). The specific dosage and dosage schedule of the additional therapeutic agent can further vary, and the optimal dose, dosing schedule and route of administration will be determined based upon the specific therapeutic agent that is being used.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammalI and calicheamicin phill, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylomithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a combination therapy of the invention may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, the LAG3 antagonist is administered before administration of the PD-1 antagonist, while in other embodiments, the LAG3 antagonist is administered after administration of the PD-1 antagonist. In another embodiment, the LAG3 antagonist is administered concurrently with the PD-1 antagonist.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

Each small molecule therapeutic agent in a combination therapy of the invention can be administered orally or parenterally, including the intravenous, intramuscular, intraperitoneal, subcutaneous, rectal, topical, and transdermal routes of administration.

A combination therapy of the invention may be used prior to or following surgery to remove a tumor and may be used prior to, during or after radiation therapy.

In some embodiments, a combination therapy of the invention is administered to a patient who has not been previously treated with a biotherapeutic or chemotherapeutic agent, i.e., is treatment-naïve. In other embodiments, the combination therapy is administered to a patient who failed to achieve a sustained response after prior therapy with a biotherapeutic or chemotherapeutic agent, i.e., is treatment-experienced.

A combination therapy of the invention is typically used to treat a tumor that is large enough to be found by palpation or by imaging techniques well known in the art, such as MRI, ultrasound, or CAT scan.

A combination therapy of the invention is preferably administered to a human patient who has a non-MSI-H or pMMR colorectal cancer that tests positive for one or both of PD-L1 and PD-L2, and preferably tests positive for PD-L1 expression. In some preferred embodiments, PD-L1 expression is detected using a diagnostic anti-human PD-L1 antibody, or antigen binding fragment thereof, in an IHC assay on an FFPE or frozen tissue section of a tumor sample removed from the patient. Typically, the patient's physician would order a diagnostic test to determine PD-L1 expression in a tumor tissue sample removed from the patient prior to initiation of treatment with the PD-1 antagonist and the LAG3 antagonist, but it is envisioned that the physician could order the first or subsequent diagnostic tests at any time after initiation of treatment, such as for example after completion of a treatment cycle. In one embodiment, the PD-L1 expression is measured by the PD-L1 IHC 22C3 pharmDx assay. In another embodiment, the patient has a Mononuclear Inflammatory Density Score for PD-L1 expression≥2. In another embodiment, the patient has a Mononuclear Inflammatory Density Score for PD-L1 expression≥3. In another embodiment, the patient has a Mononuclear Inflammatory Density Score for PD-L1 expression≥4. In another embodiment, the patient has a Tumor Proportion Score for PD-L1 expression≥1%. In another embodiment, the patient has a Tumor Proportion Score for PD-L1 expression≥10%. In another embodiment, the patient has a Tumor Proportion Score for PD-L1 expression≥20%. In another embodiment, the patient has a Tumor Proportion Score for PD-L1 expression≥30%. In a further embodiment, the patient has a Combined Positive Score for PD-L1 expression≥1%. In a further embodiment, the patient has a Combined Positive Score for PD-L1 expression between 1 and 20%. In a further embodiment, the patient has a Combined Positive Score for PD-L1 expression≥2%. In a further embodiment, the patient has a Combined Positive Score for PD-L1 expression≥5%. In yet a further embodiment, the patient has a Combined Positive Score for PD-L1 expression≥10%. In a further embodiment, the patient has a Combined Positive Score for PD-L1 expression≥15%. In yet a further embodiment, the patient has a Combined Positive Score for PD-L1 expression≥20%.

Selecting a dosage regimen (also referred to herein as an administration regimen) for a combination therapy of the invention depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Biotherapeutic agents in a combination therapy of the invention may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144.

In some embodiments that employ an anti-human PD-1 mAb as the PD-1 antagonist in the combination therapy, the dosing regimen will comprise administering the anti-human PD-1 mAb at a dose of 1, 2, 3, 5 or 10 mg/kg at intervals of about 14 days (±2 days) or about 21 days (±2 days) or about 30 days (±2 days) throughout the course of treatment.

In other embodiments that employ an anti-human PD-1 mAb as the PD-1 antagonist in the combination therapy, the dosing regimen will comprise administering the anti-human PD-1 mAb at a dose of from about 0.005 mg/kg to about 10 mg/kg, with intra-patient dose escalation.

In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose.

In certain embodiments, a subject will be administered an intravenous (IV) infusion of a medicament comprising any of the PD-1 antagonists described herein.

In one preferred embodiment of the invention, the PD-1 antagonist in the combination therapy is nivolumab, which is administered intravenously at a dose selected from the group consisting of: 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg/kg Q3W.

In another preferred embodiment of the invention, the PD-1 antagonist in the combination therapy is pembrolizumab, or a pembrolizumab variant, which is administered in a liquid medicament at a dose selected from the group consisting of 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, 10 mg/kg Q3W and flat-dose equivalents of any of these doses, i.e., such as 200 mg Q3W. In some embodiments, pembrolizumab is provided as a liquid medicament which comprises 25 mg/ml pembrolizumab, 7% (w/v) sucrose, 0.02% (w/v) polysorbate 80 in 10 mM histidine buffer pH 5.5. In other embodiments, pembrolizumab is provided as a liquid medicament which comprises about 125 to about 200 mg/mL of pembrolizumab, or antigen binding fragment thereof; about 10 mM histidine buffer; about 10 mM L-methionine, or a pharmaceutically acceptable salt thereof; about 7% (w/v) sucrose; and about 0.02% (w/v) polysorbate 80.

In some embodiments, the selected dose of pembrolizumab is administered by IV infusion. In one embodiment, the selected dose of pembrolizumab is administered by IV infusion over a time period of between 25 and 40 minutes, or about 30 minutes.

In some embodiments, the patient is treated with the combination therapy for at least 24 weeks, e.g., eight 3-week cycles. In some embodiments, treatment with the combination therapy continues until the patient exhibits evidence of PD or a CR.

The present invention also provides a medicament which comprises a PD-1 or LAG3 antagonist as described above and a pharmaceutically acceptable excipient. When the PD-1 antagonist or LAG3 antagonist is a biotherapeutic agent, e.g., a mAb, the antagonist may be produced in CHO cells using conventional cell culture and recovery/purification technologies.

Pharmaceutically acceptable excipients of the present disclosure include for instance, solvents, bulking agents, buffering agents, tonicity adjusting agents, and preservatives (see, e.g., Pramanick et al., Pharma Times, 45:65-77, 2013). In some embodiments the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent). The pharmaceutical compositions of the present disclosure are suitable for parenteral administration.

In some embodiments, the pharmaceutical compositions comprise an aqueous vehicle as a solvent. Suitable vehicles include for instance sterile water, saline solution, phosphate buffered saline, and Ringer's solution. In some embodiments, the composition is isotonic.

The pharmaceutical compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 4 to 9. In some embodiments, the pH is greater than (lower limit) 4, 5, 6, 7 or 8. In some embodiments, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical compositions may comprise a preservative. Suitable preservatives include for instance antioxidants and antimicrobial agents. However, in preferred embodiments, the pharmaceutical composition is prepared under sterile conditions and is in a single use container, and thus does not necessitate inclusion of a preservative.

In some embodiments, a medicament comprising an anti-PD-1 antibody as the PD-1 antagonist may be provided as a liquid formulation or prepared by reconstituting a lyophilized powder with sterile water for injection prior to use. WO 2012/135408 describes the preparation of liquid and lyophilized medicaments comprising pembrolizumab that are suitable for use in the present invention. In some embodiments, a medicament comprising pembrolizumab is provided in a glass vial which contains about 100 mg of pembrolizumab in 4 ml of solution. Each 1 mL of solution contains 25 mg of pembrolizumab and is formulated in: L-histidine (1.55 mg), polysorbate 80 (0.2 mg), sucrose (70 mg), and Water for Injection, USP. The solution requires dilution for IV infusion.

In some embodiments, a medicament comprising an anti-LAG3 antibody as the LAG3 antagonist may be provided as a liquid formulation or prepared by reconstituting a lyophilized powder with sterile water for injection prior to use. In one embodiment, the liquid formulation comprises about 25 mg/mL anti-LAG3 antibody; about 50 mg/mL sucrose; about 0.2 mg/mL polysorbate 80; about 10 mM L-histidine buffer at about pH 5.8-6.0; about 70 mM L-Arginine-HCl thereof; and optionally about 10 mM L-methionine.

The medicaments described herein may be provided as a kit which comprises a first container and a second container and a package insert. The first container contains at least one dose of a medicament comprising a PD-1 antagonist, the second container contains at least one dose of a medicament comprising a LAG3 antagonist, and the package insert, or label, which comprises instructions for treating a patient for non-MSI-H or pMMR colorectal cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. In some preferred embodiments of the kit, the PD-1 antagonist is an anti-PD-1 antibody and the instructions state that the medicaments are intended for use in treating a patient having a non-MSI-H or pMMR colorectal cancer that tests positive for PD-L1 expression by an IHC assay.

In other aspects, the medicament is a co-formulation of anti-LAG3 antibodies or antigen binding fragments and anti-PD-1 antibodies or antigen binding fragments with arginine or a pharmaceutically acceptable salt thereof at a total concentration of 10-1000 mM, and a buffer at pH about 5-8, and optionally 3-100 mM of methionine. In one embodiment, the co-formulation comprises about 10 to 120 mg/mL of an anti-LAG3 antibody; about 10 to 120 mg/mL of an anti-PD-1 antibody; about 30 to 120 mg/mL sucrose or trehalose; about 0.05 to 2 mg/mL polysorbate 80; about 3 to 30 mM L-histidine buffer at pH about 5.0-6.5; about 40 to 150 mM L-arginine or a pharmaceutically acceptable salt thereof and optionally, about 5 to 70 mM L-methionine.

These and other aspects of the invention, including the exemplary specific embodiments listed below, will be apparent from the teachings contained herein.

### Exemplary Specific Embodiments of the Invention

1. A LAG3 antagonist for use in the treatment of non-microsatellite instability-high (non-MSI-H) or proficient mismatch repair (pMMR) colorectal cancer, wherein the use is in combination with a PD-1 antagonist.
2. The LAG3 antagonist for use of embodiment 1, wherein the PD-1 antagonist is a monoclonal antibody, or an antigen binding fragment thereof.
3. The LAG3 antagonist for use of embodiment 1, wherein the individual is a human and the PD-1 antagonist is a monoclonal antibody, or an antigen binding fragment thereof, which specifically binds to human PD-1 and blocks the binding of human PD-L1 to human PD-1.
4. The LAG3 antagonist for use of embodiment 3, wherein the PD-1 antagonist also blocks binding of human PD-L2 to human PD-1.
5. The LAG3 antagonist for use of embodiment 4, wherein the PD-1 antagonist is a monoclonal antibody, or antigen binding fragment thereof, which comprises: (a) light chain CDRs of SEQ ID NOs: 1, 2 and 3 and (b) heavy chain CDRs of SEQ ID NOs: 6, 7 and 8.
6. The LAG3 antagonist for use of embodiment 4, wherein the PD-1 antagonist is an anti-PD-1 monoclonal antibody which comprises a heavy chain and a light chain, and wherein the heavy chain comprises a heavy chain variable region comprising SEQ ID NO:9 and the light chain comprises a light chain variable region comprising SEQ ID NO: 4.
7. The LAG3 antagonist for use of embodiment 4, wherein the PD-1 antagonist is an anti-PD-1 monoclonal antibody which comprises a heavy chain and a light chain, and wherein the heavy chain comprises SEQ ID NO:10 and the light chain comprises SEQ ID NO:5.
8. The LAG3 antagonist for use of embodiment 4, wherein the PD-1 antagonist is pembrolizumab.
9. The LAG3 antagonist for use of embodiment 4, wherein the PD-1 antagonist is a pembrolizumab variant.
10. The LAG3 antagonist for use of embodiment 4, wherein the PD-1 antagonist is nivolumab.
11. The LAG3 antagonist for use of any one of embodiments 1 to 10, wherein the LAG3 antagonist is a monoclonal antibody, or an antigen binding fragment thereof that blocks the binding of LAG3 to MHC Class II molecules.
12. The LAG3 antagonist for use of any one of embodiments 1 to 10, wherein the LAG3 antagonist is an antibody, or antigen binding fragment thereof, which comprises: (a) light chain CDRs of SEQ ID NOs: 26, 27 and 28 and (b) heavy chain CDRs of SEQ ID NOs: 29, 30 and 31.
13. The LAG3 antagonist for use of any one of embodiments 1 to 10, wherein the LAG3 antagonist is an anti-LAG3 antibody which comprises a heavy chain and a light chain, and wherein the heavy chain comprises a heavy chain variable region comprising SEQ ID NO:25 and the light chain comprises a light chain variable region comprising SEQ ID NO: 24.
14. The LAG3 antagonist for use of any one of embodiments 1 to 10, wherein the LAG3 antagonist is an anti-LAG3 antibody which comprises a heavy chain and a light chain, and wherein the heavy chain comprises SEQ ID NO:23 and the light chain comprises SEQ ID NO:22.
15. The LAG3 antagonist for use of any one of embodiments 1 to 10, wherein the LAG3 antagonist is an Ab6 variant.
16. The LAG3 antagonist for use of any one of embodiments 1 to 10, wherein the LAG3 antagonist is relatlimab.
17. The LAG3 antagonist for use of embodiment 1, wherein the PD-1 antagonist is a humanized anti-PD-1 antibody that comprises a heavy chain and a light chain, and wherein the heavy chain comprises a heavy chain variable region comprising heavy chain CDRs of SEQ ID NOs: 6, 7 and 8 and the light chain comprises a light chain variable region comprising light chain CDRs of SEQ ID NOs: 1, 2 and 3; and the LAG3 antagonist is a humanized anti-LAG3 antibody which comprises a heavy chain and a light chain, and wherein the heavy chain comprises a heavy chain variable region comprising heavy chain CDRs of SEQ ID NOs: 29, 30 and 31 and the light chain comprises a light chain variable region comprising light chain CDRs of SEQ ID NOs: 26, 27 and 28.
18. The LAG3 antagonist for use of embodiment 1, wherein the PD-1 antagonist is an anti-PD-1 antibody that comprises a heavy chain and a light chain, and wherein the heavy chain comprises a heavy chain variable region comprising SEQ ID NO:9 and the light chain comprises a light chain variable region comprising SEQ ID NO: 4; and the LAG3 antagonist is an anti-LAG3 antibody which comprises a heavy chain and a light chain, and wherein the heavy chain comprises a heavy chain variable region comprising SEQ ID NO:25 and the light chain comprises a light chain variable region comprising SEQ ID NO: 24.
19. The LAG3 antagonist for use of embodiment 1, wherein the PD-1 antagonist is an anti-PD-1 antibody that comprises a heavy chain and a light chain, and wherein the heavy chain comprises SEQ ID NO:10 and the light chain comprises SEQ ID NO: 5; and the LAG3 antagonist is an anti-LAG3 antibody which comprises a heavy chain and a light chain, and wherein the heavy chain comprises SEQ ID NO:23 and the light chain comprises SEQ ID NO: 22.
20. The LAG3 antagonist for use of any one of embodiments 1 to 19, wherein the PD-1 antagonist and LAG3 antagonist are co-formulated.
21. The LAG3 antagonist for use of any one of embodiments 1 to 19, wherein the PD-1 antagonist and LAG3 antagonist are co-administered.
22. The LAG3 antagonist for use of any one of embodiments 1 to 21, wherein the individual has not been previously treated with anti-PD-1 or anti-PD-L1 therapy or is confirmed progressive while receiving prior anti-PD-1 therapy.
23. The LAG3 antagonist for use of any one of embodiments 1 to 22, wherein the tumor cells of the individual is PD-L1 expression positive.
24. The LAG3 antagonist for use of any one of embodiments 1 to 22, wherein the individual has a Mononuclear Inflammatory Density Score for PD-L1 expression≥2.
25. The LAG3 antagonist for use of any one of embodiments 1 to 22, wherein the individual has a Combined Positive Score for PD-L1 expression≥1%.
26. The LAG3 antagonist for use of embodiment 24 or 25, wherein the PD-L1 expression is measured by the PD-L1 IHC 22C3 pharmDx assay.
27. The LAG3 antagonist for use of any one of embodiments 1-26, further comprising administering mFOLFOX7 (Oxaliplatin, Leucovorin and 5-FU) or FOLFIRI (Irinotecan, Leucovorin and 5-FU).

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fuse with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### EXAMPLES

### Example 1

### Clinical Studies of Anti-LAG3 Antibody in Advanced Solid Tumors

This is a multisite, open-label, dose-escalation study of anti-LAG3 antibody Ab6 monotherapy (Part A, Arm 1) and Ab6 in combination with pembrolizumab (Part A, Arm 2) followed by both nonrandomized and randomized dose confirmation of Ab6 in combination with pembrolizumab along with efficacy evaluations of Ab6 as monotherapy and in combination with pembrolizumab (Part B) in subjects with a histologically or cytologically confirmed diagnosis of advanced solid tumors.

During Part A of the study, subjects were allocated by nonrandom assignment to 1 of 2 treatment arms:
Arm 1: Ab6 as monotherapy escalating doses 7, 21, 70, 210 or 700 mg every 3 weeks (Q3W) via intravenous infusion (IV).
Arm 2: Ab6 escalating doses 7, 21, 70, 210 or 700 mg every 3 weeks (Q3W) IV in combination with pembrolizumab (200 mg Q3W) IV
   Part B was a dose confirmation of Ab6 in combination with pembrolizumab. Additionally, expansion cohorts assesses the antitumor efficacy of Ab6 as monotherapy and in combination with pembrolizumab. Part B consists of 4 treatment arms:
Arm 1: Ab6 as monotherapy dose 200 mg every 3 weeks (Q3W) via intravenous infusion (IV).
Arm 2: Ab6 dose 200 or 700 mg every 3 weeks (Q3W) IV in combination with pembrolizumab (200 mg Q3W) IV
Arm 3: Ab6 200 mg Q3W IV+pembrolizumab (200 mg Q3W IV)+mFOLFOX7 (oxaliplatin [85 mg/m²], leucovorin [calcium folinate, 400 mg/m²], fluorouracil [5-FU, 2400 mg/m² over 46 to 48 hours] every 2 weeks [Q2W])
Arm 4: Ab6+pembrolizumab (200 mg Q3W)+FOLFIRI (irinotecan [180 mg/m²], leucovorin [calcium folinate, 400 mg/m²], 5-FU [2400 mg/m² over 46 to 48 hours] Q2W)
Arm 5: Ab6A (400 mg [co-formulated fixed dose combination 200 mg Ab6+200 mg pembrolizumab] Q3W)

TABLE 4

| | | | | | |
|---|---|---|---|---|---|
| | Dose/ | Dose | Route of | Regimen/Treatment | |
| Drug | Potency | Frequency | Administration | Period | Use |

Part A, Arm 1

| | | | | | |
|---|---|---|---|---|---|
| Ab6 | 7 mg | Q3W | Intravenous | Day 1 of each 21-day | Experimental |
| | 21 mg | | (IV) Infusion | cycle | |
| | 70 mg | | | | |
| | 210 mg | | | | |
| | 700 mg | | | | |

Part A, Arm 2

| | | | | | |
|---|---|---|---|---|---|
| Ab6 | 7 mg | Q3W | Intravenous | Day 1 of each 21-day | Experimental |
| | 21 mg | | (IV) Infusion | cycle | |
| | 70 mg | | | | |
| | 210 mg | | | | |
| | 700 mg | | | | |
| Pembrolizumab | 200 mg | Q3W | IV Infusion | Day 1 of each 21-day | Experimental |
| | | | | cycle | |

Part B, Arm 1

| | | | | | |
|---|---|---|---|---|---|
| Ab6 | 200 mg | Q3W | IV infusion | Day 1 of each 21-day | Experimental |
| | | | | cycle | |

Part B, Arm 2

| | | | | | |
|---|---|---|---|---|---|
| Ab6 | 200 mg | Q3W | IV infusion | Day 1 of each 21-day | Experimental |
| | 700 mg | | | cycle | |
| Pembrolizumab | 200 mg | Q3W | IV Infusion | Day 1 of each 21-day | Experimental |
| | | | | cycle | |

Part B, Arm 3

| Ab6 | | 200 mg | Q3W | IV infusion | Day 1 of each 21-day | Experimental |
|---|---|---|---|---|---|---|
| | | | | | cycle | |
| Pembrolizumab | | 200 mg | Q3W | IV Infusion | Day 1 of each 21-day | Experimental |
| | | | | | cycle | |
| mFOLFOX7 | Oxaliplatin | 85 mg/m² | Q2W | IV infusion | Odd Number Cycles: | Background |
| | | 65 mg/m² | | | Day 1, Day 15 | Therapy |
| | Leucovorin^{d} | 400 mg/m² | | IV infusion | Even Number Cycles: | |
| | (Calcium | | | | Day 8 | |
| | Folinate) | | | | | |
| | 5-FU | 2400 mg/m² | | IV infusion | | |
| | | 2000 mg/m² | | | | |

Part B, Arm 4

| Ab6 | | 200 mg | Q3W | IV infusion | Day 1 of each 21-day | Experimental |
|---|---|---|---|---|---|---|
| | | | | | cycle | |
| Pembrolizumab | | 200 mg | Q3W | IV Infusion | Day 1 of each 21-day | Experimental |
| | | | | | cycle | |
| FOLFIRI | Irinotecan | 180 mg/m² | Q2W | IV infusion | Odd Number Cycles: | Background |
| | | 150 mg/m² | | | Day 1, Day 15 | Therapy |
| | Leucovorin^{d} | 400 mg/m² | | IV infusion | Even Number Cycles: | |
| | (Calcium | | | | Day 8 | |
| | Folinate) | | | | | |
| | 5-FU | 2400 mg/m² | | IV infusion | | |
| | | 2000 mg/m² | | | | |

Arm 5

| | | | | | |
|---|---|---|---|---|---|
| Ab6A | 400 mg | Q3W | IV infusion | Day 1 of each 21-day | Experimental |
| | | | | cycle | |

^{d}Depending on local practice guidelines, levofolinate calcium (200 mg/m² Q2W) may be substituted for leucovorin.

This trial used an adaptive design based on the pre-specified criteria of dose limiting toxicity (DLT). For dose escalation (Part A, Arm 1 and Arm 2), a 3+3 dose escalation design was utilized. For dose confirmation (Part B), the toxicity probability interval (TPI) design is utilized to refine the estimate of a preliminary recommended Phase 2 dose (RPTD) from Part A, Arm 2. Additionally, Part B compares the safety and antitumor efficacy of 2 doses of Ab6 in combination with pembrolizumab.

In Part A, Arm 1 (Ab6 monotherapy), the study began with a 3+3 design to identify a preliminary maximum tolerated dose (MTD) or maximum administered dose (MAD). During 3+3 dose escalation in both arms of Part A, an initial cohort of 3 subjects were enrolled to a dose level. If none of the 3 subjects experienced a DLT during the first 21 day cycle, escalation to the next dose occurred. If 1 of the 3 subjects experienced a DLT, another 3 subjects enrolled at this dose level. If 1 DLT was observed among the 6 subjects, the dose escalation continued. If more than 1 of 3 or more than 1 of 6 subjects at a dose level developed DLTs, dose escalation was terminated, and the study proceeded at the previous dose level.

Treatment in Part A, Arm 2 (Ab6 in combination with pembrolizumab) began with a 3+3 design to identify a preliminary RPTD for Part B. The starting dose of Ab6 was at least 1 dose level below that being tested in Part A, Arm 1. A fixed dose of 200 mg pembrolizumab was used in Part A, Arm 2.

Doses of Ab6 in combination with pembrolizumab was at least 1 dose level behind the monotherapy dose, and would not exceed the MTD or MAD of Part A, Arm 1. However, once the MTD or MAD for Part A, Arm 1 was established, the dose of Ab6 in Part A, Arm 2 continued escalation up to that dose. For enrollment to the last 2 dose levels of Arm 2, all 3 (or 6) subjects in the second highest dose level completed 1 cycle of treatment and DLT evaluation before the highest dose level began enrollment.

In Part B, dose confirmation and preliminary antitumor efficacy is assessed in PD-1-treatment-naïve head and neck squamous cell cancer (HNSCC), colorectal cancer (CRC), PD-1-treatment-failure HNSCC, and gastric cancer. A TPI design is used to refine the estimate of tolerability of the preliminary RPTD of Ab6 administered in combination with pembrolizumab identified in Part A, Arm 2 using the first 14 subjects enrolled in either of the HNSCC cohorts or the CRC cohort of Arm 2. For the PD-1-treatment-naïve HNSCC and CRC cohorts of Arm 2, the antitumor efficacy of Ab6 in combination with pembrolizumab is examined using an adaptive expansion design. If an individual cohort experiences an objective response rate (ORR) of at least 20% in the first 10 subjects enrolled (ie, at least 2 of 10 subjects) as assessed by response evaluation criteria in solid tumors (RECIST) 1.1 criteria, this cohort is expanded to enroll additional subjects. The HNSCC cohort may enroll a maximum of 30 additional subjects, for a maximum total of 40 subjects. The CRC cohort may enroll a maximum of 90 additional subjects, for a maximum total of 100 subjects. An additional cohort in Arm 2 enrolls 20 subjects with HNSCC that have progressed following prior anti-PD-1/PD-L1 therapy. A final cohort for Arm 2 employs a randomized comparison of 2 doses of Ab6 in combination with a fixed dose of pembrolizumab in 80 subjects with PD-1-treatment-naive gastric cancer. This cohort initiates enrollment only after the TPI dose confirmation of the first 14 subjects in the CRC and HNSCC cohorts has completed.

Additionally, Part B assessed the antitumor efficacy of Ab6 monotherapy (Arm 1) in 2 cohorts. 20 subjects are enrolled with PD-1-treatment-naïve CRC and receives Ab6 monotherapy at the RPTD. Additionally, if antitumor activity is observed in the Arm 2 gastric cancer cohort (≥8 of 40 subjects with an objective response, irrespective of dose) an additional 20 subjects with gastric cancer are enrolled to receive Ab6 monotherapy at the RPTD. Enrollment to the Arm 1 cohorts of Part B will not begin until their corresponding Arm 2 cohorts have been fully enrolled. Subjects with confirmed disease progression per irRECIST 1.1 in Arm 1 of both Part A and Part B will be allowed to crossover to Arm 2 and receive Ab6 at the RPTD in combination with pembrolizumab.

Part B also assesses the safety and antitumor efficacy of Ab6 (at the preliminary RP2D) administered in combination with pembrolizumab and mFOLFOX7 (up to 20 subjects) or FOLFIRI (up to 20 subjects) in subjects with microsatellite stable (MSS) PD-1-treatment-naïve CRC that have received ≤1 prior line of therapy. Part B also assesses the efficacy of Ab6A in subjects with advanced PD-1-treatment-failure solid tumors including renal cell carcinoma, melanoma, gastric, NSCLC, and bladder cancer.

### Subject Inclusion Criteria

1. Part A—Have a histologically or cytologically confirmed metastatic solid tumor for which there is no available therapy that may convey clinical benefit.
   Part B—Have 1 of the following histologically or cytologically confirmed tumor types:
      a. HNSCC that is considered incurable by local therapies. Subjects should have progressed after receiving platinum-containing systemic therapy. Systemic therapy given as part of multimodal treatment for locally advanced disease is allowed. The eligible primary tumor locations are oropharynx, oral cavity, hypopharynx, and larynx. Subjects may not have a primary tumor site of nasopharynx (any histology). Subjects enrolled in the PD-1-treatment-naive HNSCC cohort may not have been treated with prior anti-PD-1/PD-L1 therapy.
         Subjects enrolled in the PD-1-treatment-failure HNSCC cohort must be refractory to an FDA approved anti-PD-1/PD-L1 monoclonal antibody (mAb) as either monotherapy or in combination with other approved checkpoint inhibitors or other therapies according to their label, defined as (subjects must meet all of the following criteria):
         i. Have received at least 2 doses of anti-PD-1/PD-L1 mAb.
         ii. Have progressive disease after anti-PD-1/PD-L1 mAb defined according to RECIST 1.1. The initial evidence of PD is to be confirmed by a second assessment, no less than 4 weeks from the date of the first documented PD, in the absence of rapid clinical progression. (Note, this determination is made by investigator. If PD is confirmed, the initial date of PD documentation will be considered the date of disease progression.)
         iii. Have documented PD within 24 weeks of the last dose of anti-PD-1/PD-L1 mAb. Patients who were re-treated with anti-PD-1/PD-L1 mAb and patients who were on maintenance with anti-PD-1/PD-L1 mAb will be allowed to enter the trial as long as there is documented PD within 24 weeks of the last treatment date (with anti-PD-1/PD-L1 mAb).
      b. Adenocarcinoma of the stomach and/or gastric-esophageal junction (GEJ) that is considered inoperable and that has received, and progressed on, at least 1 prior chemotherapy regimen or HER2/neu-targeted approved therapy (if HER2/neu-positive). In both cases, subjects must not have been treated with prior anti-PD-1/PD-L1 therapy.
      c. CRC for Arm 1 and Arm 2: CRC originating in either the colon or rectum that is locally advanced unresectable or metastatic (ie, Stage IV) and that has received, and progressed on, all available standard-of-care therapies including fluoropyrimidine, oxaliplatin, and irinotecan but has not been treated with prior anti-PD-1/PD-L1 therapy.
      d. CRC for Arm 3 and Arm 4: CRC originating in either the colon or rectum that is locally advanced unresectable or metastatic (ie, Stage IV) and has been treated with ≤1 line of systemic therapy but has not been treated with prior anti-PD-1/PD-L1 therapy. Subjects eligible to receive EGFR-targeted therapy must have previously received this treatment in order to be eligible for the study. Subjects with known MSI high or MMR deficient gastric cancer or CRC (as determined by either PCR or IHC) are excluded from participating in this study. MSI high is defined as at least 2 allelic shifts occurring among the 5 analyzed microsatellite markers as detected by PCR. MMR deficient is defined as loss of expression of at least 1 of 4 proteins (MLH1, MSH2, MSH6, and/or PMS2) by IHC. If a subject's MSI status is unknown, testing is not required to determine eligibility.
         Subjects with CRC enrolled into Arm 3 or Arm 4 are not eligible to be re-enrolled in the study on Arm 1 or Arm 2 following discontinuation.
         For Arm 5 Only:
      e. Locally advanced or metastatic urothelial carcinoma of the renal pelvis, ureter, bladder, or urethra that is transitional cell type (or mixed transitional/non-transitional if predominantly transitional) that is not suitable for local therapy with curative intent.
      f. Unresectable stage III or metastatic melanoma not amenable to local therapy (may not have a diagnosis of uveal or ocular melanoma).
      g. Advanced/unresectable or metastatic renal cell carcinoma with predominantly clear cell elements.
      h. Locally advanced unresectable or metastatic gastric or GEJ adenocarcinoma.
      i. Stage IV metastatic NSCLC (according to AJCC version 8).
         All subjects in Arm 5 must have been treated with, and progressed on, an FDA-approved anti-PD-1/PD-L1 antibody either as monotherapy or in combination with other agents according to the following criteria:
         i. Have received at least 2 doses of anti-PD-1/PD-L1 mAb.
         ii. Have progressive disease after anti-PD-1/PD-L1 mAb defined according to RECIST 1.1. The initial evidence of PD is to be confirmed by a second assessment, no less than 4 weeks from the date of the first documented PD, in the absence of rapid clinical progression.
         iii. Have documented PD within 24 weeks of the last dose of anti-PD-1/PD-L1 mAb. Patients who were re-treated with anti-PD-1/PD-L1 mAb and patients who were on maintenance with anti-PD-1/PD-L1 mAb will be allowed to enter the trial as long as there is documented PD within 24 weeks of the last treatment date (with anti-PD-1/PD-L1 mAb).
2. Have measureable disease by irRECIST 1.1 criteria.

In Part A of the study, Ab6 given as monotherapy and in combination with pembrolizumab 200 mg was well tolerated and had a manageable safety profile across all doses tested. Dose escalation proceeded to the maximum dose of 700 mg without any DLTs.

In Part A combination Arm 2, out of six patients with non-MSI-H colorectal cancer (CRC), two experienced a partial response (ORR 33%). In Part B, dose confirmation and preliminary anti-tumor efficacy was assessed in PD-1-treatment-naive non-MSI-H colorectal cancer patients for with 200 mg anti-LAG3 antibody Ab6 in combination with 200 mg pembrolizumab administered every three weeks. Of the 38 patients treated with combination therapy in the Part B study, there were 4 partial responses. The overall response rate of all non-MSI-H CRC patients (Parts A and B) treated with anti-LAG3 antibody Ab6 and pembrolizumab combination therapy is 13.6% (6/44). MSI status was tested centrally using a PCR based assay.

This is in contrast to pembrolizumab monotherapy, which has demonstrated little to no antitumor efficacy in non-MSI-H or pMMR CRC. In clinical trial KEYNOTE-016 (KN016) and KEYNOTE-028 (KN028), 18 and 22 non-MSI-H or pMMR CRC patients, respectively, were treated with pembrolizumab monotherapy. Zero of 40 patients (0/18 in KN016 and 0/22 in KN028) achieved objective response by RECIST1.1 criteria. Patients in KN-016 received pembrolizumab 10 mg/kg every 2 weeks for up to 2 years or until progression. Patients treated in KN028 received pembrolizumab 10 mg/kg every 2 weeks for up to 2 years and were required to have PD-1 positivity for Combined Positive Score of >1% of scorable cells. See O'Neil B H et. al. PLoS One. 2017; 12(12).

### Example 2

### Measurement of PD-L1 Expression Levels

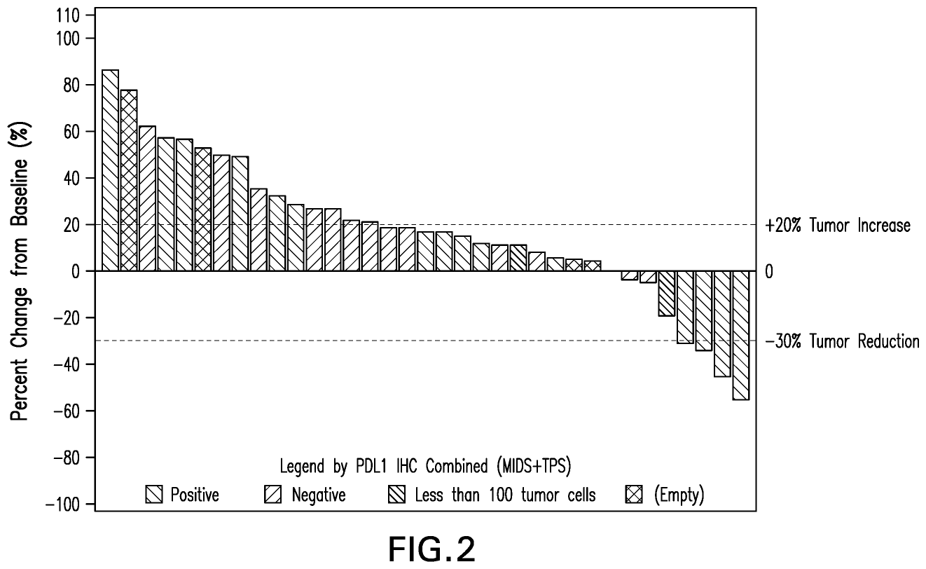

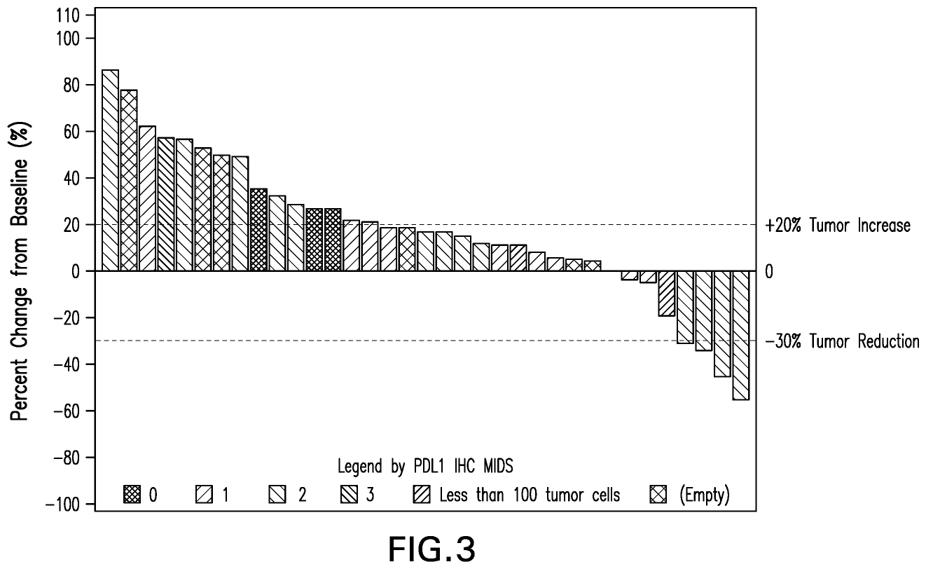

Specimens from non-MSI-H colorectal cancer patients of Part B were analyzed prior to treatment. Specimens for analysis are formalin-fixed and paraffin-embedded (FFPE) tissue sections. The IHC staining for PD-L1 expression was performed using the Dako Autostainer Link 48 platform (Dako AS480) and an automated staining protocol validated for the PD-L1 IHC 22C3 pharmDx assay according to US 2017/0285037, incorporated by reference in its entirety. The Waterfall plot of subjects with best target lesion change from baseline is shown in FIGS. 2 and 3.

FIG. 2 shows that 53% of CRC tumors in this set using the CPS scoring system are PD-L1 positive. Of the PD-L1+tumors, 4 out of 15 are responders (27%). Of the PD-L1−tumors, 0 out of 13 are responders (0%). FIG. 3 shows that using a MIDS scoring system of at least 2, of the PD-L1+tumors, 4 out of 14 are responders (28%). Of the PD-L1−tumors with a MIDS score of less than 2, 0 out of 11 are responders (0%).

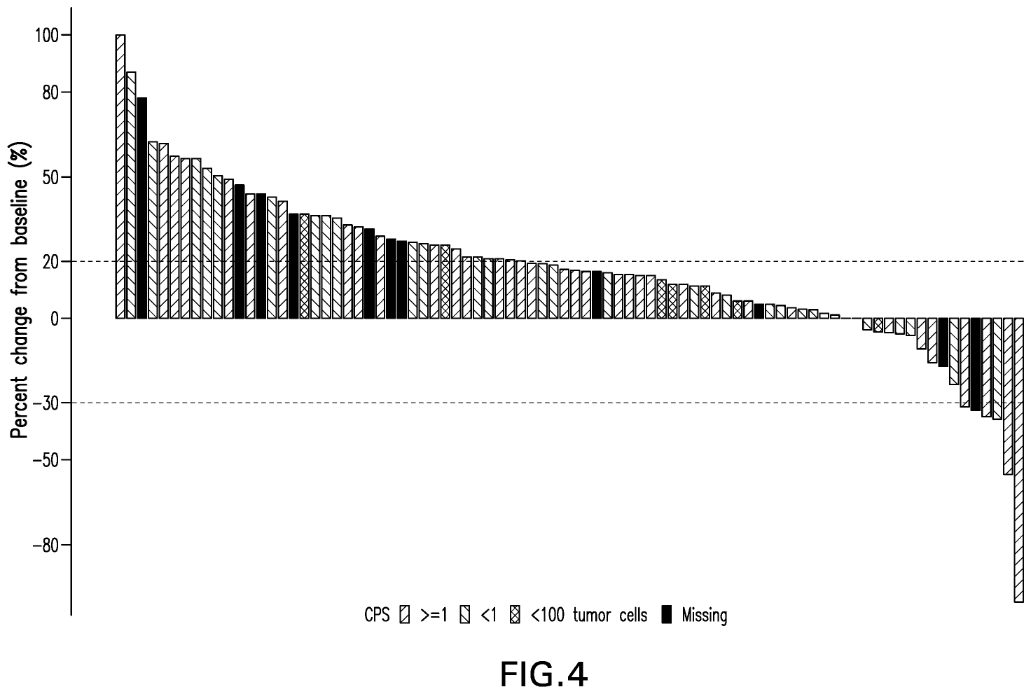

Additional IHC data was collected for an expansion cohort of Part B. FIG. 4 shows that 54% of CRC tumors in this set using the CPS scoring system are PD-L1 positive. Of the PD-L1+tumors (CPS>=1%), 4 out of 42 are responders (9%). Three responders had CPS>=1%, and 1 responder had CPS of 7%. Of the PD-L1−tumors (CPS<1%), 1 out of 35 was a responder (3%).

### REFERENCES

1. Sharpe, A. H, Wherry, E. J., Ahmed R., and Freeman G. J. The function of programmed cell death 1 and its ligands in regulating autoimmunity and infection. Nature Immunology (2007); 8:239-245.
2. Dong H et al. Tumor-associated B7-H1 promotes T-cell apoptosis: a potential mechanism of immune evasion. Nat Med. 2002 August; 8(8):793-800.
3. Yang et al. PD-1 interaction contributes to the functional suppression of T-cell responses to human uveal melanoma cells in vitro. Invest Ophthalmol Vis Sci. 2008 June; 49(6 (2008): 49: 2518-2525.
4. Ghebeh et al. The B7-H1 (PD-L1) T lymphocyte-inhibitory molecule is expressed in breast cancer patients with infiltrating ductal carcinoma: correlation with important high-risk prognostic factors. Neoplasia (2006) 8: 190-198.
5. Hamanishi J et al. Programmed cell death 1 ligand 1 and tumor-infiltrating CD8+ T lymphocytes are prognostic factors of human ovarian cancer. Proceeding of the National Academy of Sciences (2007): 104: 3360-3365.
6. Thompson R H et al. Significance of B7-H1 overexpression in kidney cancer. Clinical genitourin Cancer (2006): 5: 206-211.
7. Nomi, T. Sho, M., Akahori, T., et al. Clinical significance and therapeutic potential of the programmed death-1 ligand/programmed death-1 pathway in human pancreatic cancer. Clinical Cancer Research (2007);13:2151-2157.
8. Ohigashi Y et al. Clinical significance of programmed death-1 ligand-1 and programmed death-1 ligand 2 expression in human esophageal cancer. Clin. Cancer Research (2005): 11: 2947-2953.
9. Inman et al. PD-L1 (B7-H1) expression by urothelial carcinoma of the bladder and BCG-induced granulomata: associations with localized stage progression. Cancer (2007): 109: 1499-1505.
10. Shimauchi T et al. Augmented expression of programmed death-1 in both neoplasmatic and nonneoplastic CD4+ T-cells in adult T-cell Leukemia/Lymphoma. Int. J. Cancer (2007): 121:2585-2590.
11. Gao et al. Overexpression of PD-L1 significantly associates with tumor aggressiveness and postoperative recurrence in human hepatocellular carcinoma. Clinical Cancer Research (2009) 15: 971-979.
12. Nakanishi J. Overexpression of B7-H1 (PD-L1) significantly associates with tumor grade and postoperative prognosis in human urothelial cancers. Cancer Immunol Immunother. (2007) 56: 1173-1182.
13. Hino et al. Tumor cell expression of programmed cell death-1 is a prognostic factor for malignant melanoma. Cancer (2010): 00: 1-9.
14. Ghebeh H. Foxp3+ tregs and B7-H1+/PD-1+ T lymphocytes co-infiltrate the tumor tissues of high-risk breast cancer patients: implication for immunotherapy. BMC Cancer. 2008 Feb. 23; 8:57.
15. Ahmadzadeh M et al. Tumor antigen-specific CD8 T cells infiltrating the tumor express high levels of PD-1 and are functionally impaired. Blood (2009) 114: 1537-1544.
16. Thompson RH et al. PD-1 is expressed by tumor infiltrating cells and is associated with poor outcome for patients with renal carcinoma. Clinical Cancer Research (2007) 15: 1757-1761.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

1. A method for treating non-microsatellite instability-high (non-MSI-H) or proficient mismatch repair (pMMR) colorectal cancer in an individual comprising administering to the individual a PD-1 antagonist and a LAG3 antagonist.

2. The method of claim 1, wherein the PD-1 antagonist is a monoclonal antibody, or an antigen binding fragment thereof.

3. The method of claim 1, wherein the individual is a human and the PD-1 antagonist is a monoclonal antibody, or an antigen binding fragment thereof, which specifically binds to human PD-1 and blocks the binding of human PD-L1 to human PD-1.

4. The method of claim 3, wherein the PD-1 antagonist also blocks binding of human PD-L2 to human PD-1.

5. The method of claim 4, wherein the PD-1 antagonist is an antibody, or antigen binding fragment thereof, which comprises: (a) light chain CDRs of SEQ ID NOs: 1, 2 and 3 and (b) heavy chain CDRs of SEQ ID NOs: 6, 7 and 8.

6. The method of claim 4, wherein the PD-1 antagonist is an anti-PD-1 antibody which comprises a heavy chain and a light chain, and wherein the heavy chain comprises a heavy chain variable region comprising SEQ ID NO:9 and the light chain comprises a light chain variable region comprising SEQ ID NO: 4.

7. The method of claim 4, wherein the PD-1 antagonist is an anti-PD-1 antibody which comprises a heavy chain and a light chain, and wherein the heavy chain comprises SEQ ID NO:10 and the light chain comprises SEQ ID NO:5.

8. The method of claim 4, wherein the PD-1 antagonist is pembrolizumab.

9. The method of claim 4, wherein the PD-1 antagonist is a pembrolizumab variant.

10. The method of claim 4, wherein the PD-1 antagonist is nivolumab.

11. The method of claim 4, wherein the LAG3 antagonist is a monoclonal antibody, or an antigen binding fragment thereof that blocks binding of LAG3 to MHC Class II molecules.

12. The method of claim 5, wherein the LAG3 antagonist is an antibody, or antigen binding fragment thereof, which comprises: (a) light chain CDRs of SEQ ID NOs: 26, 27 and 28 and (b) heavy chain CDRs of SEQ ID NOs: 29, 30 and 31.

13. The method of claim 6, wherein the LAG3 antagonist is an anti-LAG3 monoclonal antibody which comprises a heavy chain and a light chain, and wherein the heavy chain comprises a heavy chain variable region comprising SEQ ID NO:25 and the light chain comprises a light chain variable region comprising SEQ ID NO: 24.

14. The method of claim 7, wherein the LAG3 antagonist is an anti-LAG3 antibody which comprises a heavy chain and a light chain, and wherein the heavy chain comprises SEQ ID NO:23 and the light chain comprises SEQ ID NO:22.

15. The method of claim 8, wherein the LAG3 antagonist is an Ab6 variant.

16. The method of claim 10, wherein the LAG3 antagonist is relatlimab.

17. The method of claim 1, wherein the PD-1 antagonist is a humanized anti-PD-1 antibody that comprises a heavy chain and a light chain, and wherein the heavy chain comprises a heavy chain variable region comprising heavy chain CDRs of SEQ ID NOs: 6, 7 and 8 and the light chain comprises a light chain variable region comprising light chain CDRs of SEQ ID NOs: 1, 2 and 3; and the LAG3 antagonist is a humanized anti-LAG3 antibody which comprises a heavy chain and a light chain, and wherein the heavy chain comprises a heavy chain variable region comprising heavy chain CDRs of SEQ ID NOs: 29, 30 and 31 and the light chain comprises a light chain variable region comprising light chain CDRs of SEQ ID NOs: 26, 27 and 28.

18. The method of claim 1, wherein the PD-1 antagonist is an anti-PD-1 antibody that comprises a heavy chain and a light chain, and wherein the heavy chain comprises a heavy chain variable region comprising SEQ ID NO:9 and the light chain comprises a light chain variable region comprising SEQ ID NO: 4; and the LAG3 antagonist is an anti-LAG3 antibody which comprises a heavy chain and a light chain, and wherein the heavy chain comprises a heavy chain variable region comprising SEQ ID NO:25 and the light chain comprises a light chain variable region comprising SEQ ID NO: 24.

19. The method of claim 1, wherein the PD-1 antagonist is an anti-PD-1 antibody that comprises a heavy chain and a light chain, and wherein the heavy chain comprises SEQ ID NO:10 and the light chain comprises SEQ ID NO: 5; and the LAG3 antagonist is an anti-LAG3 antibody which comprises a heavy chain and a light chain, and wherein the heavy chain comprises SEQ ID NO:23 and the light chain comprises SEQ ID NO: 22.

20. The method of claim 19, wherein the PD-1 antagonist and LAG3 antagonist are co-formulated.

21. The method of claim 19, wherein the PD-1 antagonist and LAG3 antagonist are co-administered.

22. The method of claim 11, wherein the individual has not been previously treated with anti-PD-1 or anti-PD-L 1 therapy or is confirmed progressive while receiving prior anti-PD-1 therapy.

23. The method of claim 1, wherein the tumor cells of the individual is PD-L1 expression positive.

24. The method of claim 1, wherein the individual has a Mononuclear Inflammatory Density Score for PD-L1 expression≥2.

25. The method of claim 1, wherein the individual has a Combined Positive Score for PD-L1 expression≥1%.

26. The method of claim 34, wherein the PD-L1 expression is measured by the PD-L1 IHC 22C3 pharmDx assay.

27. The method of claim 4, wherein the tumor cells of the individual is PD-L1 expression positive.

28. The method of claim 11, wherein the tumor cells of the individual is PD-L1 expression positive.

29. The method of claim 16, wherein the tumor cells of the individual is PD-L1 expression positive.

30. The method of claim 11, wherein the individual has a Mononuclear Inflammatory Density Score for PD-L1 expression≥2.

31. The method of claim 16, wherein the individual has a Mononuclear Inflammatory Density Score for PD-L1 expression≥2.

32. The method of claim 11, wherein the individual has a Combined Positive Score for PD-L1 expression≥1%.

33. The method of claim 16, wherein the individual has a Combined Positive Score for PD-L1 expression≥1%.

34. The method of claim 18, wherein the individual has a Combined Positive Score for PD-L1 expression≥1%.

35. The method of claim 19, wherein the individual has a Combined Positive Score for PD-L1 expression≥1%.

36. The method of claim 35, wherein the PD-L1 expression is measured by the PD-L1 IHC 22C3 pharmDx assay.
